SEQUENCE LISTING (20 sequences)

SEQ ID NO: 1 - DNA, 1116 bp, Homo sapiens
  misc_feature: InventorARAI, Tomoyuki; OKAMOTO, Yoshitaka; SAKURAI, Daiju
  misc_feature: CRLF2 transcript variant 1 cDNA
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

SEQ ID NO: 2 - protein, 371 aa, Homo sapiens
  MISC_FEATURE: CRLF2 transcript variant 1
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 3 - DNA, 780 bp, Homo sapiens
  misc_feature: CRLF2 transcript variant 2 cDNA
atggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

SEQ ID NO: 4 - protein, 259 aa, Homo sapiens
  MISC_FEATURE: CRLF2 transcript variant 2
MVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 5 - DNA, 1026 bp, Homo sapiens
  misc_feature: ETV7 transcript variant 1 cDNA
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgcatccagcccgcactgtggagcagggaggacgtgctgcactggctgcgctgggcagagcaggagtactctctgccatgcaccgcggagcacgggttcgagatgaacggacgcgccctctgcatcctcaccaaggacgacttccggcaccgtgcgcccagctcaggtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 6 - protein, 341 aa, Homo sapiens
  MISC_FEATURE: ETV7 transcript variant 1
MQEGELAISPISPVAAMPPLGTHVQARCEAQINLLGEGGICKLPGRLRIQPALWSREDVLHWLRWAEQEYSLPCTAEHGFEMNGRALCILTKDDFRHRAPSSGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 7 - DNA, 954 bp, Homo sapiens
  misc_feature: ETV7 transcript variant 2 cDNA
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgcatccagcccgcactgtggagcagggaggacgtgctgcactggctgcgctgggcagagcaggagtactctctgccatgcaccgcggagcacgggttcgagatgaacggacgcgccctctgcatcctcaccaaggacgacttccggcaccgtgcgcccagctcaggtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagaaatggacttcagctgatcttcatattcatatggagtttccagtga

SEQ ID NO: 8 - protein, 317 aa, Homo sapiens
  MISC_FEATURE: ETV7 transcript variant 2
MQEGELAISPISPVAAMPPLGTHVQARCEAQINLLGEGGICKLPGRLRIQPALWSREDVLHWLRWAEQEYSLPCTAEHGFEMNGRALCILTKDDFRHRAPSSGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRNGLQLIFIFIWSFQ

SEQ ID NO: 9 - DNA, 861 bp, Homo sapiens
  misc_feature: ETV7 transcript variant 3 cDNA
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 10 - protein, 286 aa, Homo sapiens
  MISC_FEATURE: ETV7 transcript variant 3
MQEGELAISPISPVAAMPPLGTHVQARCEAQINLLGEGGICKLPGRLRDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 11 - DNA, 849 bp, Homo sapiens
  misc_feature: ETV7 transcript variant 4 cDNA
atgcaccgcggagcacgggttcgagatgaacggacgcgccctctgcatcctcaccaaggacgacttccggcaccgtgcgcccagctcaggtcagggaaacgcacaggctttcctctaagcgggaaccctggtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 12 - protein, 282 aa, Homo sapiens
  MISC_FEATURE: ETV7 transcript variant 4
MHRGARVRDERTRPLHPHQGRLPAPCAQLRSGKRTGFPLSGNPGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 13 - DNA, 795 bp, Homo sapiens
  misc_feature: ETV7 transcript variant 5 cDNA
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgcatccagcccgcactgtggagcagggaggacgtgctgcactggctgcgctgggcagagcaggagtactctctgccatgcaccgcggagcacgggttcgagatgaacggacgcgccctctgcatcctcaccaaggacgacttccggcaccgtgcgcccagctcaggtgacgtcctgtatgagctgctccagtacatcaagacccagcggcgagccctggtgtgtgggcccttttttggagggatcttcaggctgaagacgcccacccagcactctccagtccccccggaagactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 14 - protein, 264 aa, Homo sapiens
  MISC_FEATURE: ETV7 transcript variant 5
MQEGELAISPISPVAAMPPLGTHVQARCEAQINLLGEGGICKLPGRLRIQPALWSREDVLHWLRWAEQEYSLPCTAEHGFEMNGRALCILTKDDFRHRAPSSGDVLYELLQYIKTQRRALVCGPFFGGIFRLKTPTQHSPVPPEDCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 15 - DNA, 789 bp, Homo sapiens
  misc_feature: ETV7 transcript variant 6 cDNA
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagaaatggacttcagctgatcttcatattcatatggagtttccagtga

SEQ ID NO: 16 - protein, 262 aa, Homo sapiens
  MISC_FEATURE: ETV7 transcript variant 6
MQEGELAISPISPVAAMPPLGTHVQARCEAQINLLGEGGICKLPGRLRDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRNGLQLIFIFIWSFQ

SEQ ID NO: 17 - DNA, 783 bp, Homo sapiens
  misc_feature: ETV7 transcript variant 7 cDNA
atgaacggacgcgccctctgcatcctcaccaaggacgacttccggcaccgtgcgcccagctcaggtgacgtcctgtatgagctgctccagtacatcaagacccagcggcgagccctggtgtgtgggcccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 18 - protein, 260 aa, Homo sapiens
  MISC_FEATURE: ETV7 transcript variant 7
MNGRALCILTKDDFRHRAPSSGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 19 - DNA, 573 bp, Homo sapiens
  misc_feature: ETV7 transcript variant 8 cDNA
atggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 20 - protein, 190 aa, Homo sapiens
  MISC_FEATURE: ETV7 transcript variant 8
MDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

(19) United States
(12) Patent Application Publication
(10) Pub. No.: US 2019/0093164 A1
(43) Pub. Date: Mar. 28, 2019

(54) BIOMARKER FOR DIAGNOSIS OF ALLERGIC RHINITIS

(71) Applicant: National University Corporation Chiba University, Chiba (JP)

(72) Inventors: Tomoyuki Arai, Chiba (JP); Yoshitaka Okamato, Chiba (JP); Daiju Sakurai, Chiba (JP)

(21) Appl. No.: 16/077,478

(22) PCT Filed: Feb. 21, 2017
(86) PCT No.: PCT/JP2017/006247

§ 371 Date: Dec. 12, 2018

(30) Foreign Application Priority Data
Feb. 22, 2016 (JP) 2016-031056

(51) Int. Cl. C12Q 1/6883

(52) U.S. Cl. CPC C12Q 1/6883; C12Q 2600/158

(57) ABSTRACT

An object of the present invention is to provide a marker for diagnosis of allergic rhinitis, etc. which can accurately diagnose whether allergic rhinitis has developed or not, or the risk of developing allergic rhinitis. It is possible to accurately diagnose whether allergic rhinitis has developed or not, or the risk of developing allergic rhinitis by using human CRLF2 (cytokine receptor-like factor 2) gene or human ETV7 (ETS variant 7) gene or human CRLF2 protein or human ETV7 protein as a biomarker and detecting increased expression of mRNA or cDNA of the gene or increased expression of the protein.

### TECHNICAL FIELD

The present invention relates to a method for collecting data for diagnosing allergic rhinitis, a kit for diagnosis of allergic rhinitis, a biomarker for diagnosing allergic rhinitis, and a method for screening a prophylactic or therapeutic agent for allergic rhinitis.

### BACKGROUND ART

Allergic rhinitis is an allergic disease in the nasal mucosa, such as sneezing, runny nose, or nasal stuffiness, resulting from the excessive response of the immune system to a foreign antigen which is supposed to be harmless. The number of patients having allergic rhinitis has increased rapidly since decades ago as lifestyles or living environments have changed. Thus, reduction in QOL (quality of life) of patients and increase in burden of medical expense have become problems. Particularly, the number of pollinosis (e.g., cedar pollinosis) patients is on the rise year by year, as with allergic rhinitis caused by house dust such as mite or mold.

The main cause of cedar pollinosis is considered to be an antigenic substance in cedar pollen, i.e., a cedar antigen (allergen). When cedar pollen dispersed into the atmosphere enter a human body, an immunoglobulin E antibody (IgE) against the cedar antigen is produced by B cells. This IgE binds to high-affinity IgE receptor FcεRI present on the cell membranes of mast cells or basophils. This state is called “sensitization”. When the cedar antigen enters the body again and cross-links the IgE on these cell membranes, mediators such as histamine or leukotriene are released (degranulation reaction) so that allergic rhinitis develops.

The treatment of allergic rhinitis is mainly performed by medication with an antiallergic agent typified by an antihistamine agent, an inhaled steroid drug, or the like. It has also been suggested that initial therapy of administering an antiallergic drug before pollen dispersion (pre-seasonal treatment) exhibits better findings or milder symptoms than those of treatment started after the start of dispersion or after exacerbation of symptoms.

The symptoms of allergic rhinitis are also seen in infections by viruses, bacteria, or the like. Therefore, for performing the suitable treatment of allergic rhinitis, it is important to precisely determine whether or not allergic rhinitis has developed. Meanwhile, for preventing development of allergic rhinitis, it is necessary to diagnose or predict the risk of developing allergic rhinitis by diagnosing sensitized non-developed state which is a stage prior to the development of allergic rhinitis. However, mechanisms underlying the absence of sensitization leading to sensitization and sensitization leading to the development of allergic rhinitis are poorly elucidated. Under the current circumstance, there is no effective means for diagnosing or predicting the risk of developing allergic rhinitis.

In recent years, a protein analysis technique has developed drastically and enabled comprehensive obtainment of information on (change in) protein levels in the living tissues of affected patients or the like. By use of this technique, filaggrin or apolipoprotein A-IV has been reported as a biomarker for evaluating an amelioration level of allergic rhinitis (patent documents 1 and 2). However, such a biomarker neither diagnoses nor predicts whether allergic rhinitis has developed or not, and the risk of developing allergic rhinitis.

Meanwhile, CRLF2 (cytokine receptor-like factor 2) is a protein that forms a heterodimer with an IL-7Rα chain to function as a TSLP (thymic stromal lymphopoietin) receptor (TSLPR). In recent years, it has been reported that TSLPR (CRLF2) is expressed in human peripheral basophils (non-patent document 1). It has also been reported that the expression of CRLF2 in allergic asthma patient-derived basophils is increased by allergen stimulation (non-patent document 2). However, the relation of increased expression of CRLF2 to whether allergic rhinitis has developed or not, or the risk of developing allergic rhinitis has not been known.

### PRIOR ART DOCUMENTS

### Patent Documents

Patent document 1: Japanese unexamined Patent Application Publication No. 2014-76009
Patent document 2: Japanese unexamined Patent Application Publication No. 2009-210420

### Non-Patent Documents

Non-patent document 1: Giacomin, P. R. et al., J. Immunol. 2012, 189: 4371-4378
Non-patent document 2: Agrawal, R. et al., Clin. Exp. Allergy 2014, 44: 1266-1273

### SUMMARY OF THE INVENTION

### Object to be Solved by the Invention

An object of the present invention is to provide a marker for diagnosis of allergic rhinitis, etc. which can accurately diagnose whether allergic rhinitis has developed or not, or the risk of developing allergic rhinitis.

### Means to Solve the Object

To attain the object, the present inventors have analyzed change in gene expression level in biological samples collected from sensitization-negative non-developed individuals, sensitization-positive non-developed individuals of allergic rhinitis, and sensitization-positive developed individuals of allergic rhinitis, and consequently completed the present invention by finding a marker for diagnosis of allergic rhinitis which can accurately discriminate whether allergic rhinitis has developed or not and sensitization-positive allergic rhinitis-developed state from sensitization-positive allergic rhinitis-non-developed state.

Specifically, the present invention is as follows:

[1] A method for collecting data for diagnosing allergic rhinitis, comprising detecting increased expression of mRNA or cDNA of human CRLF2 gene or ETV7 gene, or increased expression of a protein encoded by human CRLF2 gene or ETV7 gene.
[2] The method according to [1] described above, wherein increased expression of mRNAs or cDNAs of human CRLF2 gene and ETV7 gene, or increased expression of proteins encoded by human CRLF2 gene and ETV7 gene is detected.
[3] The method according to [1] or [2] described above, wherein the allergic rhinitis is cedar pollinosis or mite allergic rhinitis.
[4] The method according to any one of [1] to [3] described above, wherein the mRNA, the cDNA and the protein are basophil-derived mRNA, cDNA and protein.
[5] The method according to any one of [1] to [4] described above, further comprising detecting increased expression of allergen-specific IgE.
[6] A kit for diagnosis of allergic rhinitis, comprising primers or a probe, or a labeled form thereof for detecting expression of mRNA or cDNA of human CRLF2 gene or ETV7 gene.
[7] The kit according to [6] described above, wherein the kit comprises primers or a probe, or a labeled form thereof for detecting expression of mRNA or cDNA of human CRLF2 gene, and primers or a probe, or a labeled form thereof for detecting expression of mRNA or cDNA of human ETV7 gene.
[8] A kit for diagnosis of allergic rhinitis, comprising an antibody or a labeled form thereof specifically binding to a protein encoded by human CRLF2 gene or ETV7 gene.
[9] The kit according to [8] described above, wherein the kit comprises an antibody or a labeled form thereof specifically binding to a protein encoded by human CRLF2 gene, and an antibody or a labeled form thereof specifically binding to a protein encoded by human ETV7 gene.
[10] The kit according to any one of [6] to [9] described above, wherein the allergic rhinitis is cedar pollinosis or mite allergic rhinitis.
[11] A biomarker for diagnosing allergic rhinitis, consisting of human CRLF2 gene or ETV7 gene, or a protein encoded by human CRLF2 gene or ETV7 gene.
[12] The biomarker according to [11] described above, wherein the biomarker consists of human CRLF2 gene and human ETV7 gene, or consists of a protein encoded by human CRLF2 gene and a protein encoded by human ETV7 gene.
[13] The biomarker according to [11] or [12] described above, wherein the allergic rhinitis is cedar pollinosis or mite allergic rhinitis.
[14] A method for screening a prophylactic or therapeutic agent for allergic rhinitis, comprising the following steps (a) and (b):
(a) a step of administering a test agent or a test substance to an allergic rhinitis nonhuman animal; and
(b) a step of detecting decreased expression of mRNA or cDNA of CRLF2 gene or ETV7 gene, or decreased expression of a protein encoded by CRLF2 gene or ETV7 gene, in a biological sample collected from the nonhuman animal.
[15] The method according to [14] described above, wherein in the step (b), decreased expression of mRNAs or cDNAs of CRLF2 gene and ETV7 gene, or decreased expression of proteins encoded by CRLF2 gene and ETV7 gene is detected.
[16] The method according to [14] or [15] described above, wherein the allergic rhinitis is cedar pollinosis or mite allergic rhinitis.

Examples of other aspects of the method for screening of the present invention can include a method for determining the effectiveness of a prophylactic or therapeutic agent (drug) for allergic rhinitis, and a method for screening a prophylactic agent (drug) candidate or a therapeutic agent (drug) candidate for allergic rhinitis.

Examples of other embodiments of the present invention can include a method for diagnosing allergic rhinitis, comprising detecting increased expression of mRNA or cDNA of human CRLF2 gene or ETV7 gene, or increased expression of a protein encoded by human CRLF2 gene or ETV7 gene, and mRNA or cDNA of human CRLF2 gene or ETV7 gene, or a protein encoded by human CRLF2 gene or ETV7 gene for use in diagnosis of allergic rhinitis.

### Effect of the Invention

According to the collection method of the present invention, it is possible to accurately diagnose (discriminate) whether allergic rhinitis has developed or not, or sensitization-positive developed state of allergic rhinitis and sensitization-positive non-developed status of allergic rhinitis. Therefore, before development of allergic rhinitis, development allergic rhinitis can be prevented, or a suitable treatment of allergic rhinitis can be performed. Furthermore, the method for screening of the present invention contributes to the development of prophylactic (protective) or therapeutic agent (drug) for allergic rhinitis.

### BRIEF DESCRIPTION OF DRAWINGS

FIG. 1A is a diagram showing results of analyzing the mRNA expression level (“FPKM value” on the ordinate) of CRLF2 gene in basophils by RNA-Seq as to 3 groups regarding cedar pollinosis (healthy group, sensitized non-developed group, and developed group). FIG. 1B is a diagram showing results of analyzing the cDNA expression level of the CRLF2 gene in basophils by quantitative PCR as to the 3 groups. “RQ” on the ordinate denotes a relative value of the expression level when the expression level for stimulation “−” of the healthy group is defined as 1. In the diagram, stimulation “−” and “+” denote the absence and presence, respectively, of stimulation with a cedar antigen. In the diagram, “*”, “**”, and “***” each represent that statistically significant difference is present (p<0.05, p<0.01, and p<0.001, respectively).

FIG. 2A is a diagram showing results of analyzing the mRNA expression level (“FPKM value” on the ordinate) of ETV7 gene in basophils by RNA-Seq as to the 3 groups. FIG. 2B is a diagram showing results of analyzing the cDNA expression level of the ETV7 gene in basophils by quantitative PCR as to the 3 groups. “RQ” on the ordinate denotes a relative value of the expression level when the expression level for stimulation “−” of the healthy group is defined as 1. In the diagram, stimulation “−” and “+” denote the absence and presence, respectively, of stimulation with a cedar antigen. In the diagram, “*” represents that statistically significant difference is present (p<0.05).

FIG. 3 is a diagram showing results of analyzing the expression level of TSLPR in basophils using a flow cytometer as to the 3 groups. “Fold change in TSLPR” on the ordinate denotes the ratio of the TSLPR expression level with cedar antigen stimulation to the TSLPR expression level without cedar antigen stimulation.

FIG. 4 is a diagram showing results of determining 2 groups regarding cedar pollinosis (non-developed group [healthy group and sensitized non-developed group] and developed group) on the basis of a cedar-specific IgE antibody value (FIG. 4A), and results of determining both the groups on the basis of the expression level of TSLPR in basophils (FIG. 4B). In the diagram, the solid line depicts a mean, and the dotted line depicts a threshold. In the diagram, “*” represents that statistically significant difference is present (p<0.0001).

FIG. 5 is a diagram showing results of combining a method for determining 2 groups regarding cedar pollinosis (non-developed group and developed group) on the basis of a cedar-specific IgE antibody value (FIG. 5A), and a method for determining both the groups on the basis of the expression level of TSLPR in basophils (FIG. 5B). In the diagram, the solid line depicts a mean, and the dotted line depicts a threshold. In the diagram, “*” represents that statistically significant difference is present (p<0.01).

FIG. 6 is a diagram showing results of determining 2 groups regarding mite allergic rhinitis (non-developed group [healthy group and sensitized non-developed group] and developed group) on the basis of a mite-specific IgE antibody value (FIG. 6A), and results of determining both the groups on the basis of the expression level of TSLPR in basophils (FIG. 6B). In the diagram, the solid line depicts a mean, and the dotted line depicts a threshold. In the diagram, “*” represents that statistically significant difference is present (p<0.0001).

FIG. 7 is a diagram showing results of combining a method for determining 2 groups regarding mite allergic rhinitis (non-developed group and developed group) on the basis of a mite-specific IgE antibody value (FIG. 7A), and a method for determining both the groups on the basis of the expression level of TSLPR in basophils (FIG. 7B). In the diagram, the solid line depicts a mean, and the dotted line depicts a threshold. In the diagram, “*” represents that statistically significant difference is present (p<0.05).

### MODE OF CARRYING OUT THE INVENTION

The method for collecting data for diagnosing allergic rhinitis according to the present invention is not particularly limited as long as it is a method for collecting data for diagnosis of allergic rhinitis, comprising detecting, and optionally quantifying, increased expression of mRNA of human CRLF2 (cytokine receptor-like factor 2) gene or a reverse transcript (cDNA) thereof in a biological sample collected from a subject (provider), increased expression of mRNA or cDNA of human ETV7 (ETS variant 7) gene in the biological sample, increased expression of a protein encoded by human CRLF2 gene (human CRLF2 protein) in the biological sample, or increased expression of a protein encoded by human ETV7 gene (human ETV7 protein) in the biological sample (hereinafter, referred to as the “present collection method”).

The kit for diagnosis of allergic rhinitis according to the present invention is not particularly limited as long as it is a kit for use in diagnosis of allergic rhinitis, comprising primers or a probe, or a labeled form thereof for detecting expression of mRNA or cDNA of human CRLF2 gene in the biological sample or expression of mRNA or cDNA of human ETV7 gene in the biological sample (hereinafter, referred to as the “present kit 1 for diagnosis”), or a kit for use in diagnosis of allergic rhinitis, comprising an antibody or a labeled form thereof specifically binding to human CRLF2 protein in the biological sample, or an antibody or a labeled form thereof specifically binding to human ETV7 protein in the biological sample (hereinafter, referred to as the “present kit 2 for diagnosis”). The present kits 1 and 2 for diagnosis are a use invention relating to a kit for diagnosing allergic rhinitis. These kits usually comprise a component generally used in this kind of diagnosis kit, for example, a carrier, a pH buffer, or a stabilizer as well as an attached document such as an instruction manual or an instruction for diagnosing allergic rhinitis.

The biomarker for diagnosing allergic rhinitis according to the present invention is not particularly limited as long as it is a biomarker for diagnosis of allergic rhinitis in a subject, consisting of human CRLF2 gene or ETV7 gene, or human CRLF2 protein or ETV7 protein (hereinafter, referred to as the “present biomarker”).

The method for screening a prophylactic or therapeutic agent for allergic rhinitis according to the present invention is not particularly limited as long as it is a method comprising, in the presented order: step (a) of administering a test agent or a test substance to an allergic rhinitis nonhuman animal; and the step of detecting increased expression of mRNA or cDNA of nonhuman CRLF2 gene or ETV7 gene, or increased expression of nonhuman CRLF2 protein or ETV7 protein, in a biological sample collected from the nonhuman animal (hereinafter, referred to as the “present method for screening”).

The “diagnosis of allergic rhinitis” includes determining whether allergic rhinitis has developed or not, more specifically, discrimination between sensitization-positive developed state of allergic rhinitis and sensitization-negative non-developed or sensitization-positive non-developed state of allergic rhinitis, and determining the risk of developing allergic rhinitis, more specifically, discrimination between sensitization-positive developed state of allergic rhinitis and sensitization-positive non-developed state of allergic rhinitis.

The present collection method is preferably a method wherein increased expression of mRNA or cDNA of human CRLF2 gene and increased expression of mRNA or cDNA of human ETV7 gene are simultaneously, sequentially, or individually detected, or a method wherein increased expression of human CRLF2 protein and increased expression of human ETV7 protein are simultaneously, sequentially, or individually detected, from the viewpoint of providing data for diagnosis with higher accuracy.

The present kit 1 for diagnosis is preferably a kit comprising primers or a probe, or a labeled form thereof for detecting expression of mRNA or cDNA of human CRLF2 gene, and primers or a probe, or a labeled form thereof for detecting expression of mRNA or cDNA of human ETV7 gene, from the viewpoint of providing a kit for diagnosis with higher accuracy. The present kit 2 for diagnosis is preferably a kit comprising an antibody or a labeled form thereof specifically binding to human CRLF2 protein, and an antibody or a labeled form thereof specifically binding to human ETV7 protein, from the viewpoint of providing a kit for diagnosis with higher accuracy.

The present biomarker preferably consists of a human CRLF2 gene and ETV7 gene, or consists of human CRLF2 protein and ETV7 protein from the viewpoint of providing a biomarker for diagnosis with higher accuracy.

The present method for screening is preferably a method wherein in the step (b) described above, decreased expression of mRNA or cDNA of nonhuman CRLF2 gene and decreased expression of mRNA or cDNA of nonhuman ETV7 gene are simultaneously, sequentially, or individually detected, or a method wherein decreased expression of nonhuman CRLF2 protein and decreased expression of nonhuman ETV7 protein are simultaneously, sequentially, or individually detected, from the viewpoint of screening a prophylactic or therapeutic agent for allergic rhinitis with higher efficacy.

In the present invention, the “allergic rhinitis” means type I allergic (IgE antibody-mediated allergic) inflammation in the nasal mucosa where allergic rhinitis symptoms (nasal obstruction, paroxysmal sneezing, and/or watery rhinorrhea) appear as a result of forming an antibody in vivo by the ingestion or contact of a certain extrinsic substance and causing antigen-antibody reaction by the re-ingestion or re-contact of the same extrinsic substance (allergen [antigen]).

In the present invention, the “sensitization-negative non-developed state of allergic rhinitis” means a state where increase in IgE antibody specific for the target allergen is not observed, and the allergic rhinitis symptoms described above are not observed. The “sensitization-positive non-developed state of allergic rhinitis” means a state where increase in IgE antibody specific for the target allergen is observed, and the allergic rhinitis symptoms described above are not observed. The “sensitization-positive developed-state of allergic rhinitis” means a state where increase in IgE antibody specific for the target allergen is observed, and the allergic rhinitis symptoms described above are observed.

The allergic rhinitis is classified into perennial allergic rhinitis and seasonal allergic rhinitis according to the time of onset. The perennial allergic rhinitis is capable of developing throughout a year due to a cause such as house dust (e.g., mold, fungal spores, fibers of woven fabrics, animal scales, mites, and dead insects). On the other hand, the seasonal allergic rhinitis is capable of developing at a particular time of a year due to a cause such as pollen.

Examples of the allergic rhinitis can specifically include allergic rhinitis caused by the house dust described above, cedar pollinosis, cypress pollinosis, ragweed pollinosis, rice pollinosis, zelkova pollinosis, orchard grass pollinosis, white birch pollinosis, Quercus serrata pollinosis, alder pollinosis, and Pinus pollinosis. Among them, cedar pollinosis or allergic rhinitis that develops due to mites (mite allergic rhinitis) is preferred.

Examples of the test subject can include a test subject for which the presence or absence of allergic rhinitis is unknown, such as a sensitization-positive non-developed test subject of allergic rhinitis, and a subject who may have another disease such as bacterial rhinitis or viral rhinitis. The test subject for which the presence or absence of allergic rhinitis is unknown also includes a test subject who has a history of allergic rhinitis and for which the presence or absence of allergic rhinitis is unknown at the time of a test.

Examples of the biological sample can include: a nonliquid sample such as a tissue, a cell, and an organ; a liquid sample such as blood, urine, and saliva; and a sample containing basophils prepared from blood. Among them, blood or a sample containing basophils prepared from blood is preferred.

When the subject is a sensitization-negative non-developed individual of allergic rhinitis, usually, the expression level of mRNA or cDNA of the human CRLF2 gene or the expression level of the human CRLF2 protein rarely varies between a biological sample that is not stimulated with the target allergen (hereinafter, also referred to as an “unstimulated sample” for the sake of convenience) and a biological sample stimulated with the target allergen (hereinafter, also referred to as a “stimulated sample” for the sake of convenience).

Thus, in the present collection method, data for diagnosing the subject as being likely to be a sensitization-negative non-developed individual of allergic rhinitis can be collected when the expression level of mRNA or cDNA of the human CRLF2 gene rarely varies between the “unstimulated sample” and the “stimulated sample” derived from the subject, or when the expression level of the human CRLF2 protein rarely varies between the “unstimulated sample” and the “stimulated sample” derived from the subject.

When the subject is a sensitization-positive non-developed individual of allergic rhinitis, the expression level of mRNA or cDNA of the human CRLF2 gene or the expression level of the human CRLF2 protein is usually increased in the “stimulated sample” compared with the “unstimulated sample”. The relative value of the increase level of the “stimulated sample” to the “unstimulated sample” derived from the sensitization-positive non-developed individual (ratio; hereinafter, also referred to as a “CRLF2 ratio in the sensitized non-developed individual” for the sake of convenience) differs depending on the biological sample as a detection target, the concentration of the stimulating allergen, a detection method, etc. and therefore, cannot be generalized. In the case of detecting the basophil-derived human CRLF2 protein by immunological assay, the CRLF2 ratio in the sensitized non-developed individual is usually within the range of 1.01 to 1.4, and examples thereof can include 1.01 to 1.35, 1.01 to 1.3, 1.01 to 1.25, 1.01 to 1.2, 1.01 to 1.15, 1.01 to 1.1, 1.01 to 1.05, 1.05 to 1.35, 1.05 to 1.3, 1.05 to 1.25, 1.05 to 1.2, 1.05 to 1.15, 1.05 to 1.1, 1.1 to 1.35, 1.1 to 1.3, 1.1 to 1.25, 1.1 to 1.2, 1.1 to 1.15, 1.15 to 1.35, 1.15 to 1.3, 1.15 to 1.25, 1.15 to 1.20, 1.2 to 1.35, 1.2 to 1.3, 1.2 to 1.25, 1.25 to 1.35, 1.25 to 1.3, 1.3 to 1.35, 1.05 to 1.4, 1.1 to 1.4, 1.15 to 1.4, 1.2 to 1.4, 1.25 to 1.4, 1.3 to 1.4, and 1.35 to 1.4.

Thus, in the present collection method, data for diagnosing the subject as being likely to be a sensitization-positive non-developed individual of allergic rhinitis can be collected when the expression level ratio of the CRLF2 protein in the “stimulated sample” to that in the “unstimulated sample” is within the range of the “CRLF2 ratio in the sensitized non-developed individual”.

In the case of detecting the mRNA expression of the basophil-derived CRLF2 gene by RNA sequencing (RNA-Seq) analysis or in the case of detecting the cDNA expression of the basophil-derived human CRLF2 gene by quantitative PCR, the “CRLF2 ratio in the sensitized non-developed individual” is usually 1.2 or more, and examples thereof can include 1.3 or more, 1.6 or more, 1.7 or more, 2.0 or more, 2.3 or more, 2.6 or more, 3.0 or more, 3.3 or more, 3.6 or more, 4.0 or more, 4.3 or more, 4.6 or more, 5.0 or more, 5.3 or more, 5.6 or more, 6.0 or more, 6.3 or more, 6.6 or more, and 7.0 or more.

Thus, in the present collection method, data for diagnosing the subject as being likely to be a sensitization-positive non-developed individual of allergic rhinitis can be collected when the ratio of the expression level of mRNA or cDNA of the human CRLF2 gene in the “stimulated sample” derived from the subject to the expression level of mRNA or cDNA of the human CRLF2 gene in the “unstimulated sample” derived from the subject is equal to or more than the “CRLF2 ratio in the sensitized non-developed individual”.

When the subject is a developed individual of allergic rhinitis, the expression level of mRNA or cDNA of the human CRLF2 gene or the expression level of the human CRLF2 protein is usually increased in the “stimulated sample” compared with the “unstimulated sample”. The relative value of the increase level of the “stimulated sample” to the “unstimulated sample” derived from the developed individual (ratio; hereinafter, also referred to as a “CRLF2 ratio in the developed individual” for the sake of convenience) differs depending on the biological sample as a detection target, the concentration of the stimulating allergen, a detection method, etc. and therefore, cannot be generalized. In the case of detecting the basophil-derived human CRLF2 protein by immunological assay, the CRLF2 ratio in the developed individual is usually 1.05 or more, and examples thereof can include 1.1 or more, 1.15 or more, 1.2 or more, 1.25 or more, 1.3 or more, 1.35 or more, 1.4 or more, 1.45 or more, 1.5 or more, 1.55 or more, 1.6 or more, 1.65 or more, 1.7 or more, 1.75 or more, 1.8 or more, 1.85 or more, 1.9 or more, 1.95 or more, 2.0 or more, 2.05 or more, 2.1 or more, 2.15 or more, 2.2 or more, 2.25 or more, 2.3 or more, 2.35 or more, 2.4 or more, 2.45 or more, and 2.5 or more.

Thus, in the present collection method, data for diagnosing the subject as being likely to be a sensitization-positive developed individual of allergic rhinitis can be collected when the ratio of the expression level of the human CRLF2 protein in the “stimulated sample” derived from the subject to the expression level of the human CRLF2 protein in the “unstimulated sample” derived from the subject is equal to or more than the “CRLF2 ratio in the developed individual”.

In the case of detecting the mRNA expression of the basophil-derived human CRLF2 gene by RNA sequencing (RNA-Seq) analysis or in the case of detecting the cDNA expression of the basophil-derived human CRLF2 gene by quantitative PCR, the “CRLF2 ratio in the developed individual” is usually 1.2 or more, and examples thereof can include 1.3 or more, 1.6 or more, 1.9 or more, 2.0 or more, 2.3 or more, 2.6 or more, 3.0 or more, 3.3 or more, 3.6 or more, 4.0 or more, 4.3 or more, 4.6 or more, 5.0 or more, 5.3 or more, 5.6 or more, 6.0 or more, 6.3 or more, 6.6 or more, and 7.0 or more.

Thus, in the present collection method, data for diagnosing the subject as being likely to be a sensitization-positive developed individual of allergic rhinitis can be collected when the ratio of the expression level of mRNA or cDNA of the human CRLF2 gene in the “stimulated sample” derived from the subject to the expression level of mRNA or cDNA of the human CRLF2 gene in the “unstimulated sample” derived from the subject is equal to or more than the “CRLF2 ratio in the developed individual”.

In the present collection method, data for diagnosing the subject as being likely to be a sensitization-negative non-developed individual or a sensitization-positive non-developed individual of allergic rhinitis can be collected when the expression level of mRNA or cDNA of the human ETV7 gene rarely varies between the “unstimulated sample” and the “stimulated sample” derived from the subject, or when the expression level of the human ETV7 protein rarely varies between the “unstimulated sample” and the “stimulated sample” derived from the subject.

When the subject is a sensitization-positive developed individual of allergic rhinitis, the expression level of mRNA or cDNA of the human ETV7 gene or the expression level of the human ETV7 protein is usually increased in the “stimulated sample” compared with the “unstimulated sample”. The relative value of the increase level of the “stimulated sample” to the “unstimulated sample” derived from the sensitization-positive developed individual (ratio; hereinafter, also referred to as an “ETV7 ratio in the developed individual” for the sake of convenience) differs depending on the biological sample as a detection target, the concentration of the stimulating allergen, a detection method, etc. and therefore, cannot be generalized. In the case of detecting the mRNA expression of the basophil-derived human ETV7 gene by RNA-Seq analysis or in the case of detecting the cDNA expression of the basophil-derived human ETV7 gene by quantitative PCR, the ETV7 ratio in the developed individual is usually 1.2 or more, and examples thereof can include 1.5 or more, 2.0 or more, 2.5 or more, 3.0 or more, 3.5 or more, 4.0 or more, 4.5 or more, 5.0 or more, 5.5 or more, 6.0 or more, 6.5 or more, 7.0 or more, 7.5 or more, 8.0 or more, 8.5 or more, 9.0 or more, 9.5 or more, and 10 or more.

Thus, in the present collection method, data for diagnosing the subject as being likely to be a sensitization-positive developed individual of allergic rhinitis can be collected when the ratio of the expression level of mRNA or cDNA of the human CRLF2 gene in the “stimulated sample” derived from the subject to the expression level of mRNA or cDNA of the human ETV7 gene in the “unstimulated sample” derived from the subject is equal to or more than the “ETV7 ratio in the developed individual”, or when the ratio of the expression level of the human ETV7 protein in the “stimulated sample” derived from the subject to the expression level of the human ETV7 protein in the “unstimulated sample” derived from the subject is equal to or more than the “ETV7 ratio in the developed individual”.

In the present collection method, data for diagnosing the subject as being likely to be a sensitization-negative non-developed individual of allergic rhinitis can be collected when the expression levels of mRNAs or cDNAs of both the human CRLF2 and ETV7 genes rarely vary between the “unstimulated sample” and the “stimulated sample” derived from the subject.

In the present collection method, data for diagnosing the subject as being likely to be a sensitization-positive non-developed individual of allergic rhinitis can be collected when the expression level of mRNA or cDNA of the human CRLF2 gene in the “stimulated sample” relative to the “unstimulated sample” derived from the subject is increased, and the expression level of mRNA or cDNA of the human ETV7 gene in the “stimulated sample” relative to the “unstimulated sample” derived from the subject is not increased.

In the present collection method, data for diagnosing the subject as being likely to be a sensitization-positive developed individual of allergic rhinitis can be collected when the expression levels of mRNAs or cDNAs of both the human CRLF2 and ETV7 genes in the “stimulated sample” relative to the “unstimulated sample” derived from the subject are increased

In the present collection method, whether or not the expression level ratio of mRNA or cDNA of the human CRLF2 gene or ETV7 gene or the expression level ratio of the CRLF2 protein or ETV7 protein in the “stimulated sample” to that in the “unstimulated sample” is higher than a threshold (cutoff value) can also be used as an index to determine whether or not the expression of the mRNA or the cDNA, or the protein is increased. The threshold can be calculated by use of a routine method, for example, a ROC (receiver operating characteristic) curve obtained using statistical analysis software on the basis of the data on the “CRLF2 ratio in the sensitized non-developed individual” or the data on the “CRLF2 ratio in the developed individual”.

The “stimulated sample” can be prepared by culturing the biological sample in the presence of the allergen. The culture period of the biological sample is not particularly limited and is, for example, 10 minutes to 2 days, preferably 1 to 12 hours. Examples of the medium for use in the culture of the biological sample can include, but are not particularly limited to, a basal medium for animal cell culture (DMEM, EMEM, RPMI-1640, α-MEM, F-12, F-10, M-199, AIM-V, etc.) containing 5 to 20% fetal bovine serum (FBS). The concentration of the allergen in the medium is not particularly limited and is, for example, within the range of 0.01 to 1 ng/mL, preferably 0.05 to 0.2 ng/mL. The culture temperature is usually within the range of 30 to 40° C., preferably approximately 37° C. The CO₂ concentration at the time of culture is usually within the range of approximately 1 to 10%, preferably approximately 5%. The humidity at the time of culture is usually within the range of approximately 70 to 100%, preferably within the range of approximately 95 to 100%. It is preferred that the “unstimulated sample” should be prepared under the same conditions as those for the “stimulated sample” in the absence of the allergen.

Examples of the human CRLF2 gene can specifically include one or more polynucleotides selected from the following [A group polynucleotide]:

### [A Group Polynucleotide]

(1) a polynucleotide consisting of the nucleotide sequence represented by SEQ ID NO: 1 (cDNA encoding CRLF2 isoform 1 [NCBI Reference Sequence: NM_022148]), or a polynucleotide which consists of a nucleotide sequence derived from the nucleotide sequence represented by SEQ ID NO: 1 by the deletion, substitution and/or addition of one or several nucleotides, and whose expression is increased in a subject compared with a control individual; and
(2) a polynucleotide consisting of the nucleotide sequence represented by SEQ ID NO: 3 (cDNA encoding CRLF2 isoform 2 [NCBI Reference Sequence: NM_001012288]), or a polynucleotide which consists of a nucleotide sequence derived from the nucleotide sequence represented by SEQ ID NO: 3 by the deletion, substitution and/or addition of one or several nucleotides, and whose expression is increased in a subject compared with a control individual.

Examples of the human CRLF2 protein can specifically include one or more proteins selected from the following [A group protein]:

### [A Group Protein]

(1) a protein consisting of the amino acid sequence represented by SEQ ID NO: 2 (CRLF2 isoform 1 [NCBI Reference Sequence: NP_071431]), or a protein which consists of an amino acid sequence derived from the amino acid sequence represented by SEQ ID NO: 2 by the deletion, substitution and/or addition of one or several amino acids, and whose expression is increased in a subject compared with a control individual; and
(2) a protein consisting of the amino acid sequence represented by SEQ ID NO: 4 (CRLF2 isoform 2 [NCBI Reference Sequence: NP_001012288]), or a protein which consists of an amino acid sequence derived from the amino acid sequence represented by SEQ ID NO: 4 by the deletion, substitution and/or addition of one or several amino acids, and whose expression is increased in a subject compared with a control individual.

Examples of the human ETV7 gene can specifically include one or more polynucleotides selected from the following [B group polynucleotide]:

### [B Group Polynucleotide]

(1) a polynucleotide consisting of the nucleotide sequence represented by SEQ ID NO: 5 (cDNA encoding ETV7 isoform 1 [NCBI Reference Sequence: NM_016135]), or a polynucleotide which consists of a nucleotide sequence derived from the nucleotide sequence represented by SEQ ID NO: 5 by the deletion, substitution and/or addition of one or several nucleotides, and whose expression is increased in a subject compared with a control individual;
(2) a polynucleotide consisting of the nucleotide sequence represented by SEQ ID NO: 7 (cDNA encoding ETV7 isoform 2 [NCBI Reference Sequence: NM_001207035]), or a polynucleotide which consists of a nucleotide sequence derived from the nucleotide sequence represented by SEQ ID NO: 7 by the deletion, substitution and/or addition of one or several nucleotides, and whose expression is increased in a subject compared with a control individual;
(3) a polynucleotide consisting of the nucleotide sequence represented by SEQ ID NO: 9 (cDNA encoding ETV7 isoform 3 [NCBI Reference Sequence: NM_001207036]), or a polynucleotide which consists of a nucleotide sequence derived from the nucleotide sequence represented by SEQ ID NO: 9 by the deletion, substitution and/or addition of one or several nucleotides, and whose expression is increased in a subject compared with a control individual;
(4) a polynucleotide consisting of the nucleotide sequence represented by SEQ ID NO: 11 (cDNA encoding ETV7 isoform 4 [NCBI Reference Sequence: NM_001207037]), or a polynucleotide which consists of a nucleotide sequence derived from the nucleotide sequence represented by SEQ ID NO: 11 by the deletion, substitution and/or addition of one or several nucleotides, and whose expression is increased in a subject compared with a control individual;
(5) a polynucleotide consisting of the nucleotide sequence represented by SEQ ID NO: 13 (cDNA encoding ETV7 isoform 5 [NCBI Reference Sequence: NM_001207038]), or a polynucleotide which consists of a nucleotide sequence derived from the nucleotide sequence represented by SEQ ID NO: 13 by the deletion, substitution and/or addition of one or several nucleotides, and whose expression is increased in a subject compared with a control individual;
(6) a polynucleotide consisting of the nucleotide sequence represented by SEQ ID NO: 15 (cDNA encoding ETV7 isoform 6 [NCBI Reference Sequence: NM_001207039]), or a polynucleotide which consists of a nucleotide sequence derived from the nucleotide sequence represented by SEQ ID NO: 15 by the deletion, substitution and/or addition of one or several nucleotides, and whose expression is increased in a subject compared with a control individual;
(7) a polynucleotide consisting of the nucleotide sequence represented by SEQ ID NO: 17 (cDNA encoding ETV7 isoform 7 [NCBI Reference Sequence: NM_001207040]), or a polynucleotide which consists of a nucleotide sequence derived from the nucleotide sequence represented by SEQ ID NO: 17 by the deletion, substitution and/or addition of one or several nucleotides, and whose expression is increased in a subject compared with a control individual; and
(8) a polynucleotide consisting of the nucleotide sequence represented by SEQ ID NO: 19 (cDNA encoding ETV7 isoform 8 [NCBI Reference Sequence: NM_001207091]), or a polynucleotide which consists of a nucleotide sequence derived from the nucleotide sequence represented by SEQ ID NO: 19 by the deletion, substitution and/or addition of one or several nucleotides, and whose expression is increased in a subject compared with a control individual.

Examples of the human ETV7 protein can specifically include one or more proteins selected from the following [B group protein]:

### [B Group Protein]

(1) a protein consisting of the amino acid sequence represented by SEQ ID NO: 6 (ETV7 isoform 1 [NCBI Reference Sequence: NP_057219]), or a protein which consists of an amino acid sequence derived from the amino acid sequence represented by SEQ ID NO: 6 by the deletion, substitution and/or addition of one or several amino acids, and whose expression is increased in a subject compared with a control individual;
(2) a protein consisting of the amino acid sequence represented by SEQ ID NO: 8 (ETV7 isoform 2 [NCBI Reference Sequence: NP_001193964]), or a protein which consists of an amino acid sequence derived from the amino acid sequence represented by SEQ ID NO: 8 by the deletion, substitution and/or addition of one or several amino acids, and whose expression is increased in a subject compared with a control individual;
(3) a protein consisting of the amino acid sequence represented by SEQ ID NO: 10 (ETV7 isoform 3 [NCBI Reference Sequence: NP_001193965]), or a protein which consists of an amino acid sequence derived from the amino acid sequence represented by SEQ ID NO: 10 by the deletion, substitution and/or addition of one or several amino acids, and whose expression is increased in a subject compared with a control individual;
(4) a protein consisting of the amino acid sequence represented by SEQ ID NO: 12 (ETV7 isoform 4 [NCBI Reference Sequence: NP_001193966]), or a protein which consists of an amino acid sequence derived from the amino acid sequence represented by SEQ ID NO: 12 by the deletion, substitution and/or addition of one or several amino acids, and whose expression is increased in a subject compared with a control individual;
(5) a protein consisting of the amino acid sequence represented by SEQ ID NO: 14 (ETV7 isoform 5 [NCBI Reference Sequence: NP_001193967]), or a protein which consists of an amino acid sequence derived from the amino acid sequence represented by SEQ ID NO: 14 by the deletion, substitution and/or addition of one or several amino acids, and whose expression is increased in a subject compared with a control individual;
(6) a protein consisting of the amino acid sequence represented by SEQ ID NO: 16 (ETV7 isoform 6 [NCBI Reference Sequence: NP_001193968]), or a protein which consists of an amino acid sequence derived from the amino acid sequence represented by SEQ ID NO: 16 by the deletion, substitution and/or addition of one or several amino acids, and whose expression is increased in a subject compared with a control individual;
(7) a protein consisting of the amino acid sequence represented by SEQ ID NO: 18 (ETV7 isoform 7 [NCBI Reference Sequence: NP_001193969]), or a protein which consists of an amino acid sequence derived from the amino acid sequence represented by SEQ ID NO: 18 by the deletion, substitution and/or addition of one or several amino acids, and whose expression is increased in a subject compared with a control individual; and
(8) a protein consisting of the amino acid sequence represented by SEQ ID NO: 20 (ETV7 isoform 8 [NCBI Reference Sequence: NP 001193970]), or a protein which consists of an amino acid sequence derived from the amino acid sequence represented by SEQ ID NO: 20 by the deletion, substitution and/or addition of one or several amino acids, and whose expression is increased in a subject compared with a control individual.

The “nucleotide sequence derived by the deletion, substitution and/or addition of one or several nucleotides” means a nucleotide sequence derived by the deletion, substitution and/or addition of the number of nucleotides usually within the range of 1 to 10, preferably within the range of 1 to 7, more preferably within the range of 1 to 6, still more preferably within the range of 1 to 5, even more preferably within the range of 1 to 4, further preferably within the range of 1 to 3, still further preferably within the range of 1 to 2, most preferably 1.

The “amino acid sequence derived by the deletion, substitution and/or addition of one or several amino acids” means an amino acid sequence derived by the deletion, substitution and/or addition of the number of amino acids usually within the range of 1 to 10, preferably within the range of 1 to 7, more preferably within the range of 1 to 6, still more preferably within the range of 1 to 5, even more preferably within the range of 1 to 4, further preferably within the range of 1 to 3, still further preferably within the range of 1 to 2, most preferably 1.

In the present collection method or the present method for screening, the method for detecting or quantifying the expression level of mRNA or cDNA of the CRLF2 or ETV7 gene may be any method as long as the method can specifically detect a portion or the whole of the mRNA or the cDNA of the CRLF2 or ETV7 gene. Examples thereof can specifically include a method of extracting or purifying total RNA from cells in the biological sample, followed by RNA-Seq analysis, a method of detecting the total RNA by Northern blotting using a probe consisting of a nucleotide sequence complementary to mRNA of the CRLF2 or ETV7 gene, a method of synthesizing a reverse transcript (cDNA) of mRNA of the CRLF2 or ETV7 gene from the total RNA using reverse transcriptase, followed by detection by quantitative PCR, such as competitive PCR or real-time PCR, using a primer pair that specifically amplifies the reverse transcript, and a method of detecting the cDNA using a microarray in which a probe for CRLF2 or ETV7 gene detection (CRLF2 or ETV7 gene cDNA labeled with a labeling material such as biotin or avidin) is immobilized on a support usable in hybridization, such as glass, silicon, or plastic.

In the present collection method or the present method for screening, the method for detecting or quantifying the expression level of the CRLF2 or ETV7 protein may be any method as long as the method can specifically detect a portion or the whole of the CRLF2 or ETV7 protein. Examples thereof can specifically include mass spectrometry of detecting peptides constituting the CRLF2 or ETV7 protein, and immunological assay using an antibody specifically recognizing the CRLF2 or ETV7 protein.

Examples of the immunological assay can preferably include immunohistochemical staining, ELISA, EIA, RIA, Western blotting, and flow cytometry. The flow cytometry can be performed using a fluorescence-activated cell sorter (FACS) using a fluorescent material (allophycocyanin [APC], phycoerythrin [PE], FITC [fluorescein isothiocyanate], Alexa Fluor 488, Alexa Fluor 647, Alexa Fluor 700, PE-Texas Red, PE-Cy5, PE-Cy7, etc.)-labeled antibody specifically binding to the CRLF2 or ETV7 protein. Since the CRLF2 protein is a cell surface receptor, the CRLF2 protein in cells such as basophils can be detected in the state of live cells. Therefore, for detecting or quantifying the expression level of the CRLF2 protein in cells such as basophils, it is preferred to use flow cytometry in consideration of convenience.

In the present collection method or the present method for screening, a method of further simultaneously, sequentially or individually detecting increased or decreased expression of allergen-specific IgE is preferred for further enhancing the reliability of the data for diagnosing allergic rhinitis. It is preferred that the method of detecting increased or decreased expression of allergen-specific IgE should be carried out, particularly, before the present collection method.

In the present collection method or the present method for screening, the method of detecting increased or decreased expression of allergen-specific IgE can be a method that can detect or quantify the expression level of allergen-specific IgE in the biological sample. Examples thereof can specifically include CAP (capsulated hydrophilic carrier polymer)-RAST (radioallergosorbent test) of contacting the biological sample with the allergen (antigen) and detecting the binding of the allergen-specific IgE in the biological sample to the allergen, a histamine release test (HRT) of detecting histamine released by the binding of an IgE receptor in mast cells or basophils to the allergen, a basophil activation test (BAT) of analyzing change in activation marker (CD203, etc.) on the surface of basophil cells by allergen stimulation using a flow cytometer, and a method of conducting analysis by reporter assay using a gene in which a reporter gene (luciferase, β galactosidase, GFP, etc.) is inserted downstream of a promoter controllable by allergen-specific IgE and a transcriptional factor NF-AT (nuclear factor-activated T cell). The biological sample in the case of detecting allergen-specific IgE is usually blood, or serum or plasma prepared from blood.

For the primers in the present kit 1 for diagnosis, the length of the primer sequence, an annealing site to cDNA, the length of the cDNA to be amplified, etc. can be appropriately selected in consideration of cDNA amplification efficiency or specificity as long as they are a set of complementary primers capable of annealing to partial sequences upstream or downstream of the mRNA or the cDNA of the human CRLF2 or ETV7 gene (referred to as a “forward primer and a reverse primer”, respectively, for the sake of convenience). For example, the length of the primer sequence is usually 10 to 100 bases long, preferably 10 to 40 bases long, more preferably 10 to 30 bases long, still more preferably 15 to 30 bases long.

The forward primer and the reverse primer are usually selected such that an amplification product derived from a polynucleotide selected from [A group polynucleotide] and [B group polynucleotide] described above, which is template DNA, is specifically formed. Specifically, when position 1 of the nucleotide sequence of the polynucleotide from [A group polynucleotide] and [B group polynucleotide] described above is defined as upstream and the last position thereof is defined as downstream, the nucleotide at the 3′ end of the forward primer is usually selected so as to anneal to at least upstream of a position to which the nucleotide at the 3′ end of the reverse primer anneals, also in consideration of the avoidance of false positive ascribable to the double-stranded DNA formation (primer dimer formation) between the forward primer and the reverse primer.

The forward primer and the reverse primer can be primers, at least a portion of which anneals (hybridizes) to a portion of the template DNA (polynucleotide selected from [A group polynucleotide] and [B group polynucleotide] described above) and which enable formation of an amplification product by PCR. In this context, “at least a portion” of the forward primer or the reverse primer usually means 60% or more of the nucleotide sequence of the forward primer or the reverse primer and means preferably 65% or more, more preferably 70% or more, still more preferably 75% or more, even more preferably 80% or more, particularly preferably 85% or more, most preferably 90% or more.

For the probe in the present kit 1 for diagnosis, the length of the probe, a hybridization site, etc. can be appropriately selected in consideration of hybridization efficiency or specificity as long as it is a probe that hybridizes to a portion or the whole of the mRNA or the cDNA of the human CRLF2 or ETV7 gene. For example, the length of the probe is usually 50 to 2000 bases long, preferably 100 to 1500 bases long, more preferably 200 to 1000 bases long, still more preferably 300 to 800 bases long.

The probe is usually selected so as to anneal (hybridize) to a polynucleotide selected from [A group polynucleotide] and [B group polynucleotide] described above, which is template DNA. In this case, the probe can be a probe, at least a portion of which can anneal (hybridize) to a portion of the template DNA (polynucleotide selected from [A group polynucleotide] and [B group polynucleotide] described above). In this context, “at least a portion” of the probe usually means 60% or more of the nucleotide sequence of the probe and means preferably 65% or more, more preferably 70% or more, still more preferably 75% or more, even more preferably 80% or more, particularly preferably 85% or more, most preferably 90% or more.

The antibody in the present kit 2 for diagnosis may be an antibody such as a monoclonal antibody, a polyclonal antibody, a human antibody, a chimeric antibody, or a humanized antibody. These also include an antibody fragment consisting of a portion of the antibody, such as F(ab′)2, Fab, diabody, Fv, ScFv, or Sc(Fv)₂.

Examples of the labeling material for the labeled form in the present kit 1 or 2 for diagnosis can include: an enzyme such as peroxidase (e.g., horseradish peroxidase), alkaline phosphatase, β-D-galactosidase, glucose oxidase, glucose-6-phosphate dehydrogenase, alcohol dehydrogenase, malate dehydrogenase, penicillinase, catalase, apoglucose oxidase, urease, luciferase and acetylcholinesterase; a fluorescent material such as a fluorescein isothiocyanate, phycobiliprotein, rare earth metal chelate, dansyl chloride and tetramethylrhodamine isothiocyanate; a fluorescence protein such as green fluorescence protein (GFP), cyan fluorescence protein (CFP), blue fluorescence protein (BFP), yellow fluorescence protein (YFP), red fluorescence protein (RFP) and luciferase; a radioisotope such as ³H, ¹⁴C, ¹²⁵I and ¹³¹I; and biotin, avidin and a chemiluminescent substance.

In the step (a) of the present method for screening, examples of the method for administering a test agent or a test substance to an allergic rhinitis nonhuman animal can specifically include parenteral administration and oral administration. Examples of the parenteral administration can include intravenous administration, administration into the small artery, intramuscular administration, intracutaneous administration, subcutaneous administration, intraperitoneal administration, intraventricular administration, intracranial administration, intranasal administration, colonic administration, and percutaneous administration.

In the step (b) of the present method for screening, the test agent or the test substance can be selected as a candidate agent or substance of a prophylactic or therapeutic agent for allergic rhinitis when the expression level of mRNA or cDNA of the nonhuman CRLF2 gene in the “stimulated sample” is decreased after administration of the test agent or the test substance or when the expression level of the nonhuman CRLF2 protein in the “stimulated sample” is decreased after administration of the test agent or the test substance. On the other hand, the test agent or the test substance can be excluded from a candidate agent or substance of a prophylactic or therapeutic agent for allergic rhinitis when the expression level of mRNA or cDNA of the nonhuman CRLF2 gene in the “stimulated sample” is not decreased after administration of the test agent or the test substance or when the expression level of the nonhuman CRLF2 protein in the “stimulated sample” is not decreased after administration of the test agent or the test substance.

In the step (b) of the present method for screening, the test agent or the test substance can be selected as a candidate agent or substance of a prophylactic or therapeutic agent for allergic rhinitis when the expression level of mRNA or cDNA of the nonhuman ETV7 gene in the “stimulated sample” is decreased after administration of the test agent or the test substance or when the expression level of the nonhuman ETV7 protein in the “stimulated sample” is decreased after administration of the test agent or the test substance. On the other hand, the test agent or the test substance can be excluded from a candidate agent or substance of a prophylactic or therapeutic agent for allergic rhinitis when the expression level of mRNA or cDNA of the nonhuman ETV7 gene is not decreased after administration of the test agent or the test substance or when the expression level of the nonhuman ETV7 protein in the “stimulated sample” is not decreased after administration of the test agent or the test substance.

The allergic rhinitis nonhuman animal in the present method for screening may be a nonhuman animal that has naturally developed allergic rhinitis or may be an allergic rhinitis model rat produced according to a method described in the document “Japanese Journal of Complementary and Alternative Medicine, Vol. 9, No. 2, September 2012: 107-113”, an allergic rhinitis model mouse produced according to a method described in the document “Haenuki, Y. et al., J. Allergy Clin. Immunol. 2012, 130: 184-194 ell”, an allergic rhinitis model animal produced according to a method described in Japanese unexamined Patent Application Publication No. 2013-70653, or a commercially available allergic rhinitis model animal, for example, an egg allergy model OVA-IgE mouse (BALB/cA-Tg(IgE-H01-4)Rin Tg(IgE-kL01-4)Rin/Jcl) or a chemical allergy model TNP-IgE mouse (BALB/cA-Tg(IgE-Hb4)Rin Tg(IgE-kLb4)Rin/Jcl) (all manufactured by CLEA Japan, Inc.). Examples of the nonhuman animal can include a nonhuman mammal such as a mouse as well as a rat, a hamster, a guinea pig, a monkey, cattle, a pig, a horse, a rabbit, sheep, a goat, a cat, and a dog.

The nonhuman animal CRLF2 gene or protein (ortholog of the human CRLF2 gene or protein) is known in mice (NCBI Gene ID: 57914), rats (NCBI Gene ID: 171499), dogs (NCBI Gene ID: 491709), cattle (NCBI Gene ID: 529792), monkeys (NCBI Gene ID: 106995136), etc.

The nonhuman animal ETV7 gene or protein (ortholog of the human ETV7 gene or protein) is known in chimpanzees (NCBI Gene ID: 747854), dogs (NCBI Gene ID: 481764), cattle (NCBI Gene ID: 529792), monkeys (NCBI Gene ID: 719151), etc.

Hereinafter, the present invention will be described more specifically with reference to Examples. However, the technical scope of the present invention is not limited by these examples.

### EXAMPLES

1. Extraction of Basophil-Derived RNA 90 mL of blood was collected from each of 22 individuals in total involving 6 sensitization-positive non-developed individuals (sensitized non-developed group) of cedar pollinosis, 11 sensitization-positive developed individuals (developed group) of cedar pollinosis, and 5 sensitization-negative non-developed individuals (healthy group) of cedar pollinosis (27- to 50-year-old 16 males and 6 females), and erythrocytes were removed using HetaSep (manufactured by STEMCELL Technologies Inc.), followed by the negative separation of basophils using EasySep Neg Human Basophil Kit (manufactured by STEMCELL Technologies Inc.). The basophils derived from these 3 groups (sensitized non-developed group, sensitized developed group, and healthy group) were each cultured in a RPMI-1640 medium containing 10% FBS for 4 hours (37° C.) in the presence and absence of a 0.1 ng/mL cedar pollen extract (manufactured by LSL Co., Ltd.). Then, total RNA was purified using miRNeasy Mini kit (manufactured by Qiagen N.V.) according to the attached protocol.

### 2. RNA Sequencing Analysis and Statistical Analysis

RNA-Seq analysis using the basophil-derived total RNA was outsourced to Kazusa DNA Research Institute. As a result of analyzing the expression level of the RNA gene among the 3 groups (sensitization-positive non-developed group, sensitized developed group, and healthy group) with or without cedar antigen (cedar pollen) stimulation by statistical approaches (multiple test [ANOVA], multiple sub effect test [Tukey's test], two-group test [paired t-test]), 12 genes including CRLF2 and ETV7 genes were identified as candidates of biomarkers for allergic rhinitis diagnosis.

### 3. Quantitative PCR

Further screening of the 12 candidate genes was performed by quantitative PCR. Quantitative PCR was performed with the basophil-derived total RNA as a template using TaqMan Gene Expression Assays (manufactured by Applied Biosystems, Inc.) according to the protocol attached to the product.

As a result, the CRLF2 and ETV7 genes were identified as biomarkers for allergic rhinitis diagnosis. GAPDH gene was used as an internal standard. Assay ID of a primer or probe solution for detection of each gene used in the quantitative PCR is as follows:

### CRLF2 (Assay ID: Hs00845692_ml)

### ETV7 (Assay ID: Hs00903229_ml)

### Gapdh (Assay ID: Hs99999905_ml)

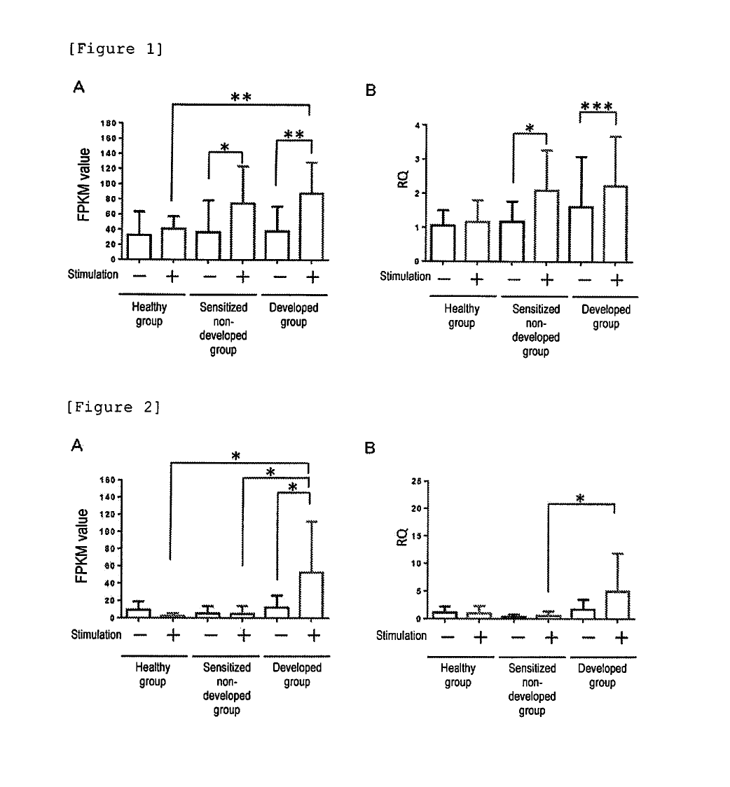

It was shown that the mRNA expression level of the CRLF2 gene was increased by 2.46±1.00 (0.45) and 2.32±1.09 (0.33) times in both the sensitized non-developed group and the developed group, respectively, by stimulation with cedar pollen compared with the case without stimulation (FIG. 1A; indicated as “mean±standard deviation (standard error)”). It was also shown that the cDNA expression level of the CRLF2 gene was also increased by 1.75±0.21 (0.09) and 1.62±0.50 (0.15) times in both the sensitized non-developed group and the developed group, respectively, by stimulation with cedar pollen compared with the case without stimulation (FIG. 1B). On the other hand, the mRNA and cDNA expression levels of the healthy group-derived CRLF2 gene rarely varied by stimulation with cedar pollen compared with the case without stimulation (FIGS. 1A and 1B).

This result indicates that sensitization-positive non-developed and sensitization-positive developed individuals of allergic rhinitis can be discriminated from sensitization-negative non-developed individuals with the presence or absence of increase in expression level by allergen stimulation as an index by analyzing the mRNA or cDNA expression of the basophil-derived CRLF2 gene by RNA-Seq analysis, quantitative PCR, or the like.

It was shown that the mRNA and cDNA expression levels of the ETV7 gene were increased by 3.12±3.04 (0.96) and 4.05±4.76 (1.80) times, respectively, in the developed group by stimulation with cedar pollen compared with the case without stimulation (FIGS. 2A and 2B). On the other hand, the mRNA and cDNA expression levels of the ETV7 gene rarely varies in both the healthy group and the sensitized non-developed group by stimulation with cedar pollen compared with the case without stimulation (FIGS. 2A and 2B).

This result indicates that sensitization-positive developed individuals of allergic rhinitis can be discriminated from sensitization-negative non-developed individuals and sensitization-positive non-developed individuals with the presence or absence of increase in expression level by allergen stimulation as an index by analyzing the mRNA or cDNA expression of the basophil-derived ETV7 gene by RNA-Seq analysis, quantitative PCR, or the like.

The results described above, taken together, indicate that sensitization-negative non-developed individuals, sensitization-positive non-developed individuals, and sensitization-positive developed individuals of allergic rhinitis can be discriminated from each other with the presence or absence of increase in expression level by allergen stimulation as an index by analyzing the mRNA or cDNA expression as to both the basophil-derived CRLF2 and ETV7 genes by RNA-Seq analysis, quantitative PCR, or the like. Specifically, a subject can be determined as being a sensitization-negative non-developed individual of allergic rhinitis when increased expression of mRNA or cDNA is observed as to neither of the CRLF2 nor ETV7 gene by allergen stimulation, a subject can be determined as being a sensitization-positive non-developed individual of allergic rhinitis when increased expression of mRNA or cDNA of the CRLF2 gene is observed by allergen stimulation, and increased expression of mRNA or cDNA of the ETV7 gene is not observed, and a subject can be determined as being a sensitization-positive symptomatic individual of allergic rhinitis when increased expression of mRNA or cDNA is observed as to both the CRLF2 and ETV7 genes by allergen stimulation.

### 4. Flow Cytometry Analysis (1)

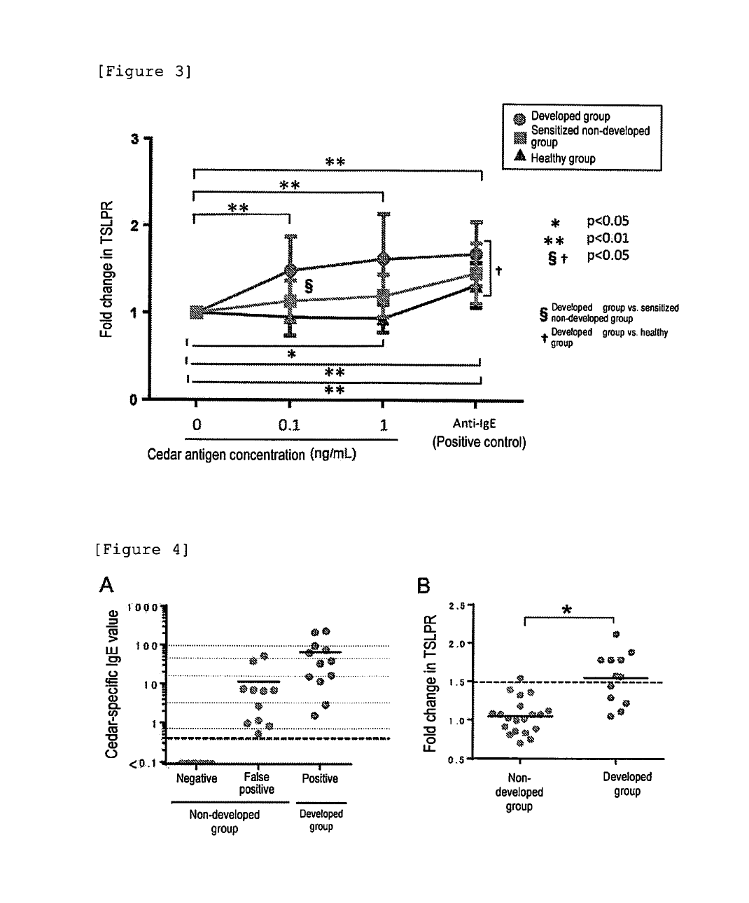

The expression of a TSLP receptor (TSLPR) constituted by the basophil-derived CRLF2 protein was analyzed using a flow cytometer. Specifically, 90 mL of blood was collected from each of 33 individuals in total involving 11 sensitization-positive non-developed individuals (sensitized non-developed group) of cedar pollinosis, 13 sensitization-positive developed individuals (developed group) of cedar pollinosis, and 9 sensitization-negative non-developed individuals (healthy group) of cedar pollinosis. After culture in a RPMI-1640 medium containing 10% FBS for 4 hours (37° C.) in the presence and absence of a 0.1 or 1 ng/mL cedar pollen extract (manufactured by LSL Co., Ltd.), antigen-antibody reaction was performed for 30 minutes under a condition of 4° C. using an anti-TSLPR antibody (manufactured by BioLegend, Inc.) labeled with a labeling material as well as 7-AAD (7-amino-actinomycin D) (manufactured by BD Biosciences) for removing dead cells, an anti-CD3 antibody (manufactured by BioLegend, Inc.) labeled with a labeling material for negative selection, and an anti-CRTH2 antibody labeled with a labeling material for positive selection. Then, the gating of basophils and the expression analysis of basophil-derived TSLPR were performed using a flow cytometer (FACS Aria II [manufactured by BD Biosciences]) (FIG. 3). An experiment of stimulating blood derived from the 3 groups (sensitized non-developed group, sensitized developed group, and healthy group) with anti-IgE was also conducted as a positive control.

It was shown that when the sensitized non-developed group-derived basophils were stimulated with 0.1 and 1 ng/mL cedar pollen, the expression level of TSLPR was increased by 1.18±0.24 and 1.29±0.46 times, respectively, as compared with the case without stimulation (FIG. 3; indicated as “mean±standard deviation”). On the other hand, it was shown that when the developed group-derived basophils were stimulated with 0.1 and 1 ng/mL cedar pollen, the expression level of TSLPR was increased by 1.43±0.33 and 1.46±0.31 times, respectively, as compared with the case without stimulation (FIG. 3).

Specifically, it was shown that although TSLPR in the basophils derived from the sensitized non-developed group and the developed group is increased by allergen stimulation, the increase level is higher in the developed group compared with the sensitized non-developed group.

On the other hand, even when the healthy group-derived basophils were stimulated with cedar pollen, the expression level of TSLPR rarely varied as compared with the case without stimulation (FIG. 3).

The results described above indicate that sensitization-negative non-developed individuals, sensitization-positive non-developed individuals, and sensitization-positive developed individuals of allergic rhinitis can be discriminated from each other with the presence or absence of increase in expression level by allergen stimulation or the increase level of the expression level as an index by analyzing the expression of basophil-derived TSLPR by immunological assay such as flow cytometry.

### 5. Flow Cytometry Analysis (2)

Next, whether or not the determination accuracy would be improved was analyzed in the case of performing the determination of a non-developed group (healthy group and sensitized non-developed group) and a developed group of cedar pollinosis by a method for detecting the expression level of basophil-derived TSLPR instead of a conventional method for detecting a cedar-specific IgE antibody value in serum by use of ImmunoCAP. Specifically, basophils derived from 20 individuals in an non-developed group (9 individuals in a healthy group and 11 individuals in a sensitized non-developed group) of cedar pollinosis negative in the nasal provocation test and basophils derived from 12 individuals in a developed group of cedar pollinosis positive in the nasal provocation test were each stimulated with a 0.1 ng/mL cedar pollen extract (manufactured by LSL Co., Ltd.) according to the method described in the preceding section “4. Flow cytometry analysis (1)”. The expression level of TSLPR was measured, and the ratio to the expression level of TSLPR without cedar pollen stimulation (fold change in TSLPR) was calculated. The ImmunoCAP and the nasal provocation test were conducted according to the methods described in the document “Practical Guideline for the Management of Allergic Rhinitis in Japan—perennial rhinitis and pollinosis—, 2016, edited by Drafting Committee for Acute Rhinosinusitis Management Guideline, the Japanese Rhinologic Society, Life Science Co., Ltd.”.

As a result, when the threshold (cutoff value) of fold change in TSLPR was set to 1.5, the proportion of true positive patients with 1.5 or more in the developed group (sensitivity) was 58.3% (7/12). The proportion of true negative patients with less than 1.5 in the non-developed group (specificity) was 95.0% (19/20). The proportion of the true positive patients and the true negative patients to the whole developed group and non-developed group (rate of correct diagnosis) was as high as 81.3% (FIG. 4B).

On the other hand, when class 1 (cedar-specific IgE antibody titer [UA/mL]: 0.35) reportedly indicating suspected positive was set as the threshold in the determination based on cedar-specific IgE, the proportion of true positive patients with class 1 or higher (0.35 [UA/mL] or more) in the developed group (sensitivity) was 100% (12/12), whereas the proportion of true negative patients with lower than class 1 (less than 0.35 [UA/mL]) in the non-developed group (specificity) was 45% (9/20). The proportion of the true positive patients and the true negative patients to the whole developed group and non-developed group (rate of correct diagnosis) was on the order of 66.0% (FIG. 4A).

This result indicates that in the case of determining the non-developed group and the developed group of cedar pollinosis, the method of the present invention based on the expression level of basophil-derived TSLPR is superior in accuracy (specificity and rate of correct diagnosis) to the conventional method based on a cedar-specific IgE antibody value.

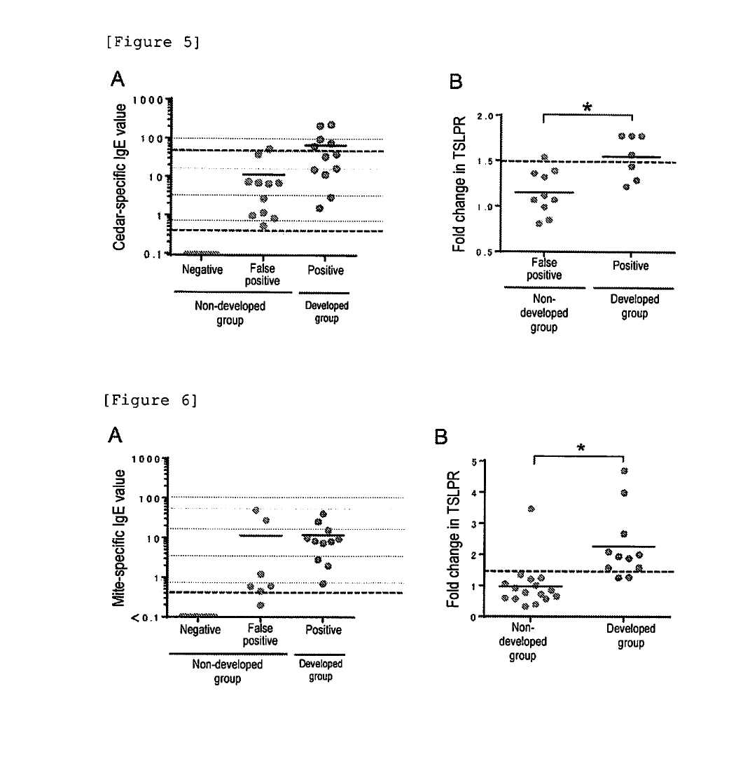

Next, the determination of the non-developed group and the developed group of cedar pollinosis was performed by combining the method based on a cedar-specific IgE antibody value, which is a conventional method, and the method of the present invention based on the expression level of basophil-derived TSLPR. Specifically, when class (cedar-specific IgE antibody titer [UA/mL]: 50; upper dotted line in FIG. 5A) was further set as the threshold for the results of FIG. 4A, the determination of 17 patients corresponding to classes 1 to 5 (patients within the range of the two dotted lines in FIG. 5A) was suspended, and the other 15 patients were determined with class 5 or higher (50 [UA/mL] or more) as positive and lower than class 1 (less than 0.35 [UA/mL]) as negative. As a result, the proportion of true positive patients with class 5 or higher in the developed group (sensitivity) was 100% (5/5). The proportion of true negative patients with lower than class 1 in the non-developed group (specificity) was 90% (9/10). The proportion of the true positive patients and the true negative patients to the whole developed group and non-developed group (rate of correct diagnosis) was 93.3% (FIG. 5A). As for the 17 patients whose determination was suspended, the determination based on the expression level of basophil-derived TSLPR was performed by setting the threshold of fold change in TSLPR to 1.5. As a result, the proportion of true positive patients with 1.5 or more in the developed group (sensitivity) was 57.1% (4/7). The proportion of true negative patients with less than 1.5 in the non-developed group (specificity) was 90% (9/10). The proportion of the true positive patients and the true negative patients to the whole developed group and non-developed group (rate of correct diagnosis) was 76.5% (FIG. 5B).

This result indicates that: by a primary determination method based on a cedar-specific IgE antibody value as a conventional method, a negative individual can be determined as being likely to be a sensitization-negative non-developed individual of cedar pollinosis, a positive individual can be determined as being likely to be a sensitization-positive developed individual of cedar pollinosis, and an individual who cannot be confirmed to be positive or negative is further subjected to a secondary determination method based on the expression level of basophil-derived TSLPR (method of the present invention); and in the secondary determination method, a positive individual can be determined as being likely to be a sensitization-positive developed individual of cedar pollinosis, and a negative individual can be determined as being likely to be a sensitization-positive non-developed individual of cedar pollinosis.

In conclusion, the conventional method and the method of the present invention are combined, whereby a cedar pollinosis patient who cannot be confirmed to be a sensitization-positive non-developed individual or a developed individual by the conventional method can be determined by the method of the present invention, and as a result, the non-developed group and the developed group of cedar pollinosis can be determined with high accuracy (rate of correct diagnosis: 84.4%, 27/32).

### 6. Flow Cytometry Analysis (3)

Next, whether or not the determination accuracy would be improved was analyzed in the case of performing the determination of a non-developed group (healthy group and sensitized non-developed group) and a developed group of mite allergic rhinitis by a method for detecting the expression level of basophil-derived TSLPR instead of a conventional method for detecting a mite-specific IgE antibody value in serum by use of ImmunoCAP. Basophils derived from 16 individuals in an non-developed group (9 individuals in a healthy group and 7 individuals in a sensitized non-developed group) of mite allergic rhinitis negative in the nasal provocation test and basophils derived from 11 individuals in a developed group of mite allergic rhinitis positive in the nasal provocation test were each stimulated with a 0.1 ng/mL mite antigen (manufactured by INDOOR biotechnologies, Inc.) according to the method described in the preceding section “4. Flow cytometry analysis (1)”. The expression level of TSLPR was measured, and the ratio to the expression level of TSLPR without mite antigen stimulation (fold change in TSLPR) was calculated. The ImmunoCAP and the nasal provocation test were conducted according to the methods described in the document “Practical Guideline for the Management of Allergic Rhinitis in Japan—perennial rhinitis and pollinosis—, 2016, edited by Drafting Committee for Acute Rhinosinusitis Management Guideline, the Japanese Rhinologic Society, Life Science Co., Ltd.”.

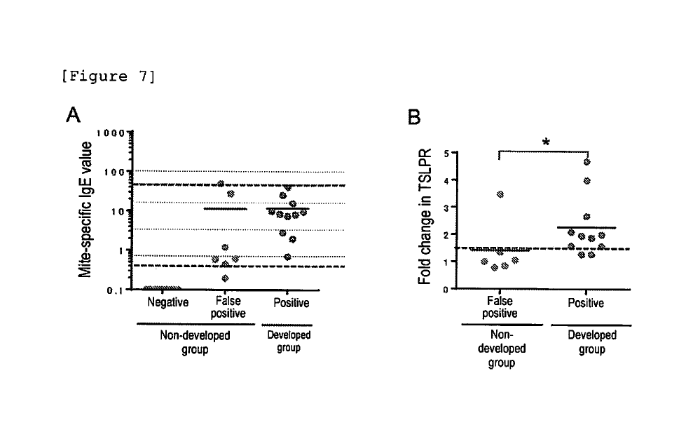

As a result, when the threshold of fold change in TSLPR was set to 1.5, the proportion of true positive patients with 1.5 or more in the developed group (sensitivity) was 81.8% (9/11). The proportion of true negative patients with less than 1.5 in the non-developed group (specificity) was 93.8% (15/16). The proportion of the true positive patients and the true negative patients to the whole developed group and non-developed group (rate of correct diagnosis) was as high as 88.9% (FIG. 6B).

On the other hand, when class 1 (mite-specific IgE antibody titer [UA/mL]: 0.35) reportedly indicating suspected positive was set as the threshold in the determination based on mite-specific IgE, the proportion of true positive patients with class 1 or higher (0.35 [UA/mL] or more) in the developed group (sensitivity) was 100% (11/11), whereas the proportion of true negative patients with lower than class 1 (less than 0.35 [UA/mL]) in the non-developed group (specificity) was 62.5% (10/16). The proportion of the true positive patients and the true negative patients to the whole developed group and non-developed group (rate of correct diagnosis) was on the order of 77.8% (FIG. 6A).

This result indicates that in the case of determining the non-developed group and the developed group of mite allergic rhinitis, the method of the present invention based on the expression level of basophil-derived TSLPR is superior in accuracy (specificity and rate of correct diagnosis) to the conventional determination method based on a mite-specific IgE antibody value.

Next, the determination of the non-developed group and the developed group of mite allergic rhinitis was performed by combining the method based on a mite-specific IgE antibody value, which is a conventional method, and the method of the present invention based on the expression level of basophil-derived TSLPR. Specifically, when class 5 (mite-specific IgE antibody titer [UA/mL]: 50; upper dotted line in FIG. 7A) was further set as the threshold for the results of FIG. 6A, the determination of 17 patients corresponding to classes 1 to 5 (patients within the range of the two dotted lines in FIG. 7A) was suspended, and the other 10 patients were determined with class 5 or higher (50 [UA/mL] or more) as positive and lower than class 1 (less than 0.35 [UA/mL]) as negative. As a result, the proportion of true negative patients with lower than class 1 in the non-developed group (specificity) was 100% (10/10). The proportion of the true negative patients to the whole non-developed group (rate of correct diagnosis) was 100% (FIG. 7A). As for the 17 patients whose determination was suspended, the determination based on the expression level of basophil-derived TSLPR was performed by setting the threshold of fold change in TSLPR to 1.5. As a result, the proportion of true positive patients with 1.5 or more in the developed group (sensitivity) was 81.8% (9/11). The proportion of true negative patients with less than 1.5 in the non-developed group (specificity) was 83.3% (5/6). The proportion of the true positive patients and the true negative patients to the whole developed group and non-developed group (rate of correct diagnosis) was 82.4% (FIG. 7B).

This result indicates that: by a primary determination method based on a mite-specific IgE antibody value as a conventional method, a negative individual can be determined as being likely to be a sensitization-negative non-developed individual of mite allergic rhinitis, and an individual who cannot be confirmed to be negative is further subjected to a secondary determination method based on the expression level of basophil-derived TSLPR (method of the present invention); and in the secondary determination method, a positive individual can be determined as being likely to be a sensitization-positive developed individual of mite allergic rhinitis, and a negative individual can be determined as being likely to be a sensitization-positive non-developed individual of mite allergic rhinitis.

In conclusion, the conventional method and the method of the present invention are combined, whereby a mite allergic rhinitis patient who cannot be confirmed to be a sensitization-positive non-developed individual or a developed individual by the conventional method can be determined by the method of the present invention, and as a result, the non-developed group and the developed group of mite allergic rhinitis can be determined with high accuracy (rate of correct diagnosis: 88.9%, 24/27).

### INDUSTRIAL APPLICABILITY

The present invention contributes to the diagnosis, prevention, and treatment of allergic rhinitis as well as the development of a prophylactic or therapeutic agent for allergic rhinitis.

1. A method for diagnosing allergic rhinitis, comprising detecting increased expression of mRNA or cDNA of either or both of human CRLF2 gene and ETV7 gene, or increased expression of a protein encoded by either or both of human CRLF2 gene and ETV7 gene in a biological sample collected from a test subject.

2. The method according to claim 1, wherein the allergic rhinitis is cedar pollinosis or mite allergic rhinitis.

3. The method according to claim 1, wherein the mRNA, the cDNA and the protein are basophil-derived mRNA, cDNA and protein.

4. The method according to claim 1, further comprising detecting increased expression of allergen-specific IgE.

5-11. (canceled)

12. The method according to claim 2, wherein the mRNA, the cDNA and the protein are basophil-derived mRNA, cDNA and protein.

13. The method according to claim 2, further comprising detecting increased expression of allergen-specific IgE.

14. The method according to claim 3, further comprising detecting increased expression of allergen-specific IgE.

15. The method according to claim 12, further comprising detecting increased expression of allergen-specific IgE.

16. The method according to claim 1, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

17. The method according to claim 2, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

18. The method according to claim 3, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

19. The method according to claim 4, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

20. The method according to claim 12, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

21. The method according to claim 13, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

22. The method according to claim 14, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.

23. The method according to claim 15, wherein the increased expression is an increase of expression level in a biological sample that is stimulated with a target allergen with respect to the expression level in a biological sample that is not stimulated with the target allergen,
wherein the test subject is diagnosed to be likely a sensitization-positive non-developed individual of allergic rhinitis or a sensitization-positive developed individual of allergic rhinitis, and wherein the method further comprises preventing onset of allergic rhinitis or treating allergic rhinitis of the test subject.
